(19) United States
(12) Patent Application Publication
(10) Pub. No.: US 2012/0204866 A1
(43) Pub. Date: Aug. 16, 2012

(54) SYSTEM AND METHODS OF INTUBATION

(76) Inventor: Robert T. Kizer, Omaha, NE (US)

(21) Appl. No.: 13/404,979

(22) Filed: Feb. 24, 2012

Related U.S. Application Data
(63) Continuation-in-part of application No. 12/380,005, filed on Feb. 20, 2009, now abandoned.
(60) Provisional application No. 61/066,703, filed on Feb. 21, 2008.

(51) Int. Cl. A61M 16/04

(52) U.S. Cl. 128/200.26

(57) ABSTRACT

The system of the present invention includes an instrument including a tube and one or more devices configured to improve patient comfort during intubation and to achieve proper tube placement in a patient's body. Embodiments of the present invention include a device configured to enter a patient's body in a collapsed state. In such embodiments, the device may achieve an erected state to facilitate positioning a tube into a patient's body.

This continuation-in-part patent application claims the benefit of U.S. Non-Provisional patent application Ser. No. 12/380,005 filed Feb. 20, 2009, which claims benefit of U.S. Provisional Patent Application Ser. No. 61/066,703 filed Feb. 21, 2008.

### FIELD OF THE INVENTION

The present invention generally relates to a medical system and methods and, more particularly, to a system and methods for intubation. The present invention is discussed in the following largely with reference to nasogastric intubation, but the present invention may be applicable to a variety of intubation procedures, for example, orogastric intubation, orotracheal intubation, or nasotracheal intubation.

### BACKGROUND OF THE INVENTION

For purposes of this application, the term “patient” may include a human or any other subject that may be intubated.

Nasogastric intubation is a medical procedure involving the placement of a tube such as a nasogastric tube (“NG tube”) into a cavity of a patient. For purposes of this application, the term “cavity” may include a nasal cavity, oral cavity, or any orifice in a patient's body. The tube may be inserted through a cavity and urged through the patient's body on a course that takes the tube through several organs. The several organs may include the nose, past the pharynx, down through the esophagus, and into and through the stomach.

NG intubation is performed to move a substance into or out of a cavity. With respect to the former, a NG tube may be used for feeding and the delivery of medication, nutrition, oxygen, or other gasses. With respect to the latter, a NG tube may be used for nasogastric aspiration, or suction, to drain contents from the stomach or small intestine. Nasogastric aspiration is mainly used to remove gastric secretions and swallowed air in a patient with a gastrointestinal obstruction. Nasogastric aspiration can be used also when a potentially toxic substance, such as poison, has been ingested or when a substance, such as alcohol, has been ingested in a potentially toxic quantity. Nasogastric aspiration is also used for preparing for a surgery and to extract samples of gastric liquid for analysis. NG tubes can also be used in association with esophageal manometry, pH monitoring, and impedance, in which the muscle pressure and amount or content of refluxed gastric contents in the esophagus are measured.

Conventional nasogastric intubation involves a multi-step process. Before a NG tube is inserted into a cavity, a health care provider, such as a nurse or doctor, must determine where the insertion end of a device will be positioned. The position may be selected based on the intended purpose of intubation such as feeding, treatment, or diagnosis. The health care provider then measures the distance from a cavity to an external body part corresponding to the site within a patient near which the insertion end of a device may be positioned. For example, a health care provider may measure the distance from the nasolabial fold to the tragus of ear or from the tip of the patient's nose to their ear and down to the xyphoid process. A health care provider may take other measurements if, for example, a particular embodiment of a tube requires an action such as swallowing.

In a conventional insertion process, a health care provider lubricates the insertion end of the NG tube, and then inserts the insertion end of the NG tube into a cavity such as the nostrils. The health care provider moves the insertion end of the NG tube through the nasal cavity and down into the throat. Once the insertion end of the NG tube is past the pharynx, the health care provider inserts the NG tube into the esophagus and stomach. The terminal end of the NG tube remains outside of the nostrils. The conventional process requires a NG tube having enough elasticity to move along the nonlinear path into the patient's body and also sufficient rigidity such that pushing the terminal end of the tube will cause the tube to progress into the patient's body and not become coiled.

During insertion, great care must be taken to ensure that the NG tube is positioned properly, and that it does not enter the trachea and move down into the lungs. The health care provider inserts the NG Tube into the nasal cavity or oral cavity and then into the pharynx. The pharynx, which is the part of the neck and throat, is situated immediately posterior to the oral cavity or mouth and nasal cavity. The pharynx includes a nasopharynx, oropharynx, and hypopharynx. The nasopharynx lies behind the nasal cavity and typically extends from the hard and soft palates to the base of the skull. The oropharynx lies behind the oral cavity. The hypopharynx, sometimes called the laryngopharynx, extends to the larynx, which is situated just below the junction that diverges into the trachea and esophagus. The esophagus leads to the stomach or, more generally, the abdomen. The trachea leads to the lungs.

A conventional process to identify proper placement of a NG tube includes injecting air into the NG tube. If the health care provider can detect air in the stomach with a stethoscope, then the NG tube is in the correct position. Another conventional process to ensure proper placement of the NG tube is to aspirate fluid from the tube with a syringe. Then, the fluid is tested with pH paper to determine the acidity of the fluid. If the pH is below a certain level, such as 5.5 pH, then the NG tube may be in the correct position. Additionally, identification of tube position may be obtained with an X-ray of the chest/abdomen. However, each of these detection methods has certain limitations including, for example, high rates of false positive results or high expense.

Certain medical procedures involve the insertion of other devices, including nasal trumpet endoscopes for nasal and laryngeal endoscopy, or surgical apparatuses for nasal, oral and laryngeal surgery, through a nose or mouth. Also, certain chemicals or medications may be applied in the nostril.

Overall, the currently available systems and methods to facilitate intubation or other related processes are not without serious shortcomings. Nasogastric intubation, positioning medical devices, chemicals, or medications in a patient's nose may cause trauma such as nose bleeds, sinusitis, and a sore throat. More significant trauma may occur including erosion of the nose where the tube is positioned, esophageal perforation, pulmonary aspiration, a collapsed lung, or intracranial placement of the NG tube. Some of these complications increase in frequency and severity the longer the NG tube is left in place. Certain of these adverse effects can result in serious complications or death of the patient.

Certain apparatuses have been developed to assist in placement of a NG tube. Some known apparatuses for assisting in placing a NG tube include a sleeve that conforms to the interior of the nasal cavity or otherwise adopt the shape of its surroundings. There are a number of disadvantages with such a configuration. Such an apparatus is likely to apply significant pressure to all sides of the cavity, which may lead to additional friction, abrasions, or ulcerations in the cavity wall. In addition, a health care provider has limited control over the apparatus shape since the shape is defined by the cavity.

Accordingly, there is a need for improved intubation system and methods that permits control over the profile of that which is inserted into the cavity in order to facilitate proper placement of the device within the patient as well as to minimize trauma and to improve patient comfort both during and after an intubation procedure. The present invention satisfies the demand.

### SUMMARY OF THE INVENTION

The present invention is directed to various embodiments by which a patient may be intubated or by which instrument placement in a cavity such as mouth or nose may be facilitated. Embodiments of the present may improve quickness and safety compared to conventional apparatuses for intubation and similar processes. Proper placement of a tube such as a NG tube may be assessed, complications minimized, and patient comfort increased both during and after a procedure. Embodiments of the present invention include facilitating positioning of a tube, a camera, medical tools, or any other object that may be beneficial to position in a cavity of a patient.

Certain embodiments of the system of the present invention include an instrument having a tube and an intubation facilitating device configured to facilitate the positioning the tube into a cavity of a patient. Certain embodiments of a device may be configured to position a tube such that an insertion end of the tube—that is, the end that is inserted into the nasal cavity—enters the throat, esophagus, stomach, or small intestine. Generally, at least a portion of another end of the tube, which is termed a “terminal end” for purposes of this application, remains outside of the patient's body such that a health care provider may maneuver that end.

Generally, an intubation facilitating device includes a body component having an inside surface and an outside surface. The inside surface may define a channel configured to permit the entry and passage of a tube. The body component also may include an input end and a receiving end. The input end may be configured to be inserted into the patient's cavity, and the receiving end may be configured to receive a tube.

Embodiments of an intubation facilitating device may include one or more of the following embodiments: the slider device, stylet device, director device, swallower device, slider stylet device, slider swallower device, slider director device, director swallower device, director stylet device, slider director stylet device, slider director swallower device, slider stylet swallower device, director stylet swallower device, and slider stylet director swallower device. For purposes of this application, the term “device” will be used to refer generally to any or all of the above listed devices and any other embodiment of an intubation facilitating device.

Certain embodiments of the system of the present invention include an instrument having a tube and a slider device. Advantageously, certain embodiments of the slider device permit a health care provider to control the profile of the slider device. Such embodiments of a slider device may be configured to achieve a collapsed state and an erected state. In a collapsed state, the slider device achieves a low profile such that the device may be inserted more easily and more comfortably into a cavity of a patient. In certain embodiments, a slider device in a collapsed state is configured to achieve and maintain the low profile before, during, and after being positioned in the patient cavity. In certain embodiments, a slider device achieves and maintains its collapsed state whether or not the device is positioned in a confined space. The slider device will likely be inserted into a cavity in a low profile. Advantageously, a maintainable low profile permits insertion of the slider device into a cavity with less friction and less contact with the cavity. In certain embodiments, the low profile is sized and shaped to permit the slider device to enter constricted portions of a body cavity.

In an erected state, the slider device achieves an expanded profile such that the NG tube may be positioned through the channel defined by the inside surface of the expanded slider device. In certain embodiments, a slider device having an expanded profile is expanded in at least a radial direction relative to the collapsed state. In certain embodiments, only a portion of the slider device achieves an erected state. In certain embodiments, a slider device in an erected state may expand to fill a cavity. In other embodiments, a slider device in an erected state has a profile expanded relative to the collapsed state, yet is configured to maintain a profile smaller than the cavity in which it is positioned without applying continual expansive pressure against the cavity wall.

The collapsed state may be achieved and maintained by a constricting component. A constricting component may have a number of embodiments. In certain embodiments, a constricting component may include a weak adhesion element which may adhere two or more points of the inside surface of a slider device. A constricting component may include a connector element which connects two or more points of the outside surface of a slider device. Certain embodiments of a constricting component may include an anchoring unit which may run the entire length of the slider device and to which the slider device may be bound. A constricting component may include one or more seal elements which define a suction compartment within the slider device. In certain embodiments, a constricting component may be made from dissolvable or digestible material such that if a portion of the constricting component is dislodged, a patient could digest the constricting component.

In certain embodiments, an erecting unit may cause a slider device to transition from a collapsed state to an erected state. Embodiments of an erecting unit are configured to disrupt a constricting component. An erecting unit may disrupt a constricting component by, for example, causing detachment of a constricting component from a slider device, causing the constricting component to break into two or more pieces, or causing the constricting component to change size, shape, physical state, or adhesiveness. In certain embodiments, an erecting unit may cause detachment of a constricting component from a slider device, for example, by putting pressure on the inside surface or outside surface of the slider device or putting pressure on the constricting component itself.

Various embodiments of an erecting unit may be used in the system of the present invention. An erecting unit may include a NG tube, a second slider device, or another device such as a stylet device, director device, or swallower device. Other embodiments of an erecting unit include a bladder element which may be positioned within a channel of a slider device and then inflated such that any constricting component is disrupted. In other embodiments, an erecting unit is a conditional component of the slider device. A conditional component causes the slider device to achieve an erected state in response to changes in environmental conditions such as temperature, moisture, humidity, alkalinity, or other environmental condition. In one embodiment, a conditional component may include a fixing agent that dissolves at a certain temperature—for example, the body temperature of a patient—such that the fixing agent no longer maintains the slider device in a collapsed state upon reaching that temperature. Embodiments of a conditional component also may include the material out of which the device is made such as a digestible material or a material that dissolves at a certain temperature or pH. Additional embodiments of a conditional component may include a sponge capable of expanding or contracting based on exposure to moisture.

In certain embodiments, a slider device may be configured to permit transfer of a substance into a patient. In such embodiments, the slider device may achieve an erected state, for example, by activation of a conditional component. In such embodiments, a health care professional may insert the slider device into the cavity, and the slider device may expand to achieve an erected state through absorption of moisture. The moisture may include natural secretions in the body cavity or may include liquids ingested for this purpose.

A slider device may be inserted into the cavity of a patient. A slider device and NG tube may be inserted simultaneously or the slider device may be inserted into the patient cavity before the NG tube. The insertion end of the NG tube may be inserted into the receiving end of a slider device. Once the slider device and NG tube are positioned in the patient cavity, the insertion end of the NG tube may be urged past the input end of the slider device. When the NG tube is in the desired position, the slider device may remain in the cavity. In other embodiments, the slider device is withdrawn while the NG tube remains in the cavity. In still other embodiments, the slider device may be separated into two or more pieces by tearing the slider device or breaking the slider device along a weakening such as a perforation and then removing the pieces from the cavity.

Another embodiment of the system of the present invention includes an instrument having a NG tube and a stylet device. One embodiment of a stylet device includes a pellet element positioned on one end of the stylet device. The pellet element may be weighted to assist in positioning the stylet device within a cavity of a patient such as a pharynx. The health care provider may use the stylet device to guide the NG tube or other device, into the pharynx by allowing the NG tube to be threaded over or within the stylet device or following alongside the stylet device.

Yet another embodiment of the system of the present invention includes an instrument having a NG tube and a director device. One embodiment of a director device includes a guide element that may be manipulated by a user to curve or angle one end or a portion of the director device for positioning the device within a cavity of the patient. In certain embodiments, a guide element includes a first guide end and a second guide end. A first guide end may be joined to the input end of the device and may be positioned through the channel such that a health care provider may access the second guide end. Then, by pulling, pushing, or otherwise manipulating the guide element, the health care provider may position the input end of the device in the cavity. Certain embodiments of a director device also include a protrusion element that is positionable to protect the curvature of a patient's cavity and may be configured to direct the path of a tube inserted through the channel. In certain embodiments, the first guide end may be attached to the protrusion element such that the position of the protrusion element may be manipulated. Upon positioning the director device, a NG tube may be inserted through the director device.

Yet another embodiment of the system of the present invention includes an instrument having a NG tube and a swallower device. One embodiment of a swallower device includes a conduit component and one or more bladder elements. A bladder element may achieve an inflated state from a deflated state when either the conduit component or the bladder element is injected with a selected substance, which may include a liquid or a gas. In certain embodiments, the liquid or gas may have a scent or flavor such that swallowing the simulated food bolus is more appetizing or otherwise easier for the patient. Embodiments of a bladder element may be linked to a conduit component via in a number of configurations. In certain embodiments, a bladder element is linked to the outside surface of the conduit component. In certain embodiments, a bladder element is integrated into the conduit component. Embodiments of a bladder element simulate a food bolus to assist the patient in advancing portions of the swallower device and portions of the NG tube into his or her stomach.

Yet another embodiment of the system of the present invention includes an instrument having a NG tube and a slider stylet device. One embodiment of a slider style device includes a body component having an input end and a receiving end. A slider stylet device may include a pellet element positioned at or near the input end and at least some portion of the device may be configured to achieve a collapsed state and an erected state. In certain embodiments, the body component and the pellet element may be configured to achieve a collapsed state and an erected state. Certain embodiments of the slider stylet device include a channel within the pellet element or adjacent to the pellet element.

Yet another embodiment of the system of the present invention includes an instrument having a NG tube and a slider director device. A slider director may include a guide element and may be configured to achieve a collapsed state and an erected state.

Yet another embodiment of the system of the present invention includes an instrument having a NG tube and a slider swallower device. Embodiments of a slider swallower device may include a bladder element and may be configured to achieve a collapsed state and an erected state. The bladder element may be positioned at or near the input end such that the bladder element simulates a food bolus that assists the patient in swallowing the device. In certain embodiments, a bladder element may be positioned largely within a channel of the slider swallower device such that inflating the bladder element causes the slider swallower device to transition from a collapsed state to an erected state.

Yet another embodiment of the system of the present invention includes an instrument having a NG tube and a director stylet device. Embodiments of a director stylet device may include a pellet element and a guide element. Certain embodiments of the director stylet device include a channel within the pellet element or adjacent to the pellet element.

Yet another embodiment of the system of the present invention includes an instrument having a NG tube and a director swallower device. Embodiments of a director swallower device may include a guide element and a bladder element.

Yet another embodiment of the system of the present invention includes an instrument having a slider tube device. Embodiments of a slider tube device may permit the passage of a substance through a channel defined by an inside surface of the device. The slider tube device may be configured to achieve a collapsed state and an erected state.

Yet embodiment of the system of the present invention includes an instrument having a NG tube and a slider director swallower device.

Embodiments of a slider director swallower device may include a guide element and may be configured to achieve a collapsed state and an erected state. In certain embodiments, a bladder element may be positioned largely within a channel of the slider director swallower device such that inflating the bladder element causes the slider director swallower device to transition from a collapsed state to an erected state.

Yet another embodiment of the system of the present invention includes an instrument having a NG tube and a slider director stylet device. Embodiments of a slider director stylet device may include a guide element, includes a pellet element, and may be configured to achieve a collapsed state and an erected state. Certain embodiments of the slider director stylet device include a channel within the pellet element or adjacent to the pellet element.

Yet another embodiment of the system of the present invention includes an instrument having a NG tube and slider stylet swallower device. Embodiments of a slider director swallower device may include a pellet element, a bladder element, and may be configured to achieve a collapsed state and an erected state. In certain embodiments, a bladder element may be positioned largely within a channel of the slider stylet swallower device such that inflating the bladder element causes the slider stylet swallower device to transition from a collapsed state to an erected state.

Yet another embodiment of the system of the present invention includes an instrument including a NG tube and a slider stylet director swallower device. Embodiments of a slider stylet director swallower device may include a guide element, a pellet element, a bladder element, and may be configured to achieve a collapsed state and an erected state. In certain embodiments, a bladder element may be positioned largely within a channel of the slider stylet director swallower device such that inflating the bladder element causes the slider stylet director swallower device to transition from a collapsed state to an erected state. Certain embodiments of the slider stylet director swallower device include a channel within the pellet element or adjacent to the pellet element.

For purposes of this application, the term “instrument” includes not only a NG tube, but also a slider device, stylet device, swallower device, director device, slider stylet device, slider swallower device, slider director device, director swallower device, director stylet device, slider director stylet device, slider director swallower device, slider stylet swallower device, director stylet swallower device, and slider stylet director swallower device. The devices according to the present invention may be used in conjunction with one another for insertion into a cavity of a patient so that a NG tube can be positioned, for example, through a nostril of the nose, past the pharynx, down through the esophagus, and into the stomach.

In one embodiment of an instrument, the director device may be used with the swallower device to insert and position a NG tube in a cavity of a patient. The director device may be inserted into the nostril and in the pharynx of a patient. The NG tube may be positioned on the swallower device, which then may be threaded within the director device.

In another embodiment of an instrument, the slider device may be used with the swallower device and NG tube. The slider device may be inserted into the nostril and in the pharynx of a patient. The NG tube may be positioned on the swallower device, which then may be threaded within the slider device and causes the slider device to achieve an erected state.

In yet another embodiment of an instrument, the slider device may be used with the director device and NG tube. The slider device may be inserted into the nostril and in the pharynx of a patient. The director device may be threaded through the slider device—causing the slider device to achieve an erected state—and properly positioned within the cavity such as the pharynx. The NG tube may be then inserted in the director device.

In another embodiment of an instrument, the stylet device and NG tube may be used with the director device. The director device may be inserted into the nostril and in the pharynx of a patient. The NG tube may be positioned on the stylet device, which then may be threaded within the director device and causes the slider device to achieve an erected state.

In another embodiment of an instrument, the stylet device may be used with the swallower device and NG tube. In certain embodiments, the stylet device may be inserted into the nostril and in the pharynx of a patient. The NG tube may be positioned on the swallower device, which then may be threaded over the stylet device and cause the slider device to achieve an erected state. Once positioned, the stylet device may be removed from the NG tube. This embodiment is contemplated for use with larger diameter NG tubes.

In another embodiment of an instrument, the stylet device and NG tube may be used with the slider device. The slider device may be inserted into the nostril and in the pharynx of a patient. The NG tube may be positioned on the stylet device, which then may be threaded within the slider device and causes the slider device to achieve an erected state.

In yet another embodiment of an instrument, the NG tube, slider device, director device, and swallower device may all be used in combination. The slider device may be inserted into the nostril and in the pharynx of a patient. The director device may be threaded through the slider device—causing the slider device to achieve an erected state—and inserted into the pharynx. The NG tube may be positioned on the swallower device, which then may be threaded within the erected slider device including director device.

In yet another embodiment of an instrument, the NG tube, slider device, director device, and stylet device may all be used in combination. The slider device may be inserted into the nostril and in the pharynx of a patient. The director device may be threaded through the slider device—causing the slider device to achieve an erected state—and properly positioned within a cavity such as the pharynx. The NG tube may be positioned on the stylet device, which then may be threaded within the slider device including director device.

In yet another embodiment, the NG tube, stylet device, slider device, and swallower device may all be used in combination. The stylet device may be inserted into the nostril and in the pharynx of a patient. The slider device is threaded over the stylet device, which causes the slider device to achieve an erected state. Once positioned, the stylet device may be removed. The NG tube is positioned on the swallower device and inserted through the erected slider device.

In yet another embodiment, the NG tube, stylet device, slider device, director device and swallower device may all be used in combination. The stylet device may be inserted into the nostril and in the pharynx of a patient. The slider device is threaded over the stylet device and causes the slider device to achieve an erected state. Once positioned, the stylet device may be removed. The director device is inserted through the erected slider device. The NG tube positioned in association with the swallower device may then be inserted into the director device. Once positioned, the director device may be removed.

In certain embodiments, one or more of the slider device, stylet device, swallower device, director device, slider stylet device, slider swallower device, slider director device, director swallower device, director stylet device, slider director stylet device, slider director swallower device, slider stylet swallower device, director stylet swallower device, and slider stylet director swallower device may be used in conjunction with another device to position a NG tube in a cavity.

Embodiments of an instrument may include a NG tube or a device having a flavor or scent such that insertion of the instrument into a cavity is made more pleasant by appealing to those senses. In certain embodiments, such a flavor or scent may be a result of the material out of which the instrument is made. In certain other embodiments, a selected flavor or scent may be added to the instrument, for example, by applying a flavor coating.

Certain embodiments of an instrument may be configured to include a detection component to assist a health care provider in determining the location of a device or a NG tube within the patient. A detection component may cause an emission such as sound, light, radio waves, magnetic field, or ultrasound waves. In such embodiments, a detector element is configured to perceive the emission to identify the location of the tube. In other embodiments, a human may perceive the emission to identify the position of the tube. One embodiment includes a NG tube with a radiofrequency identification tag (RFID) that can be read by a detector element positioned outside of the patient. Another such embodiment includes a NG tube with a light source that can emit light that, when positioned inside a patient such as in an esophagus or stomach, is perceivable by a human eye outside of the patient. Another such embodiment includes a NG tube with a metal component such that a detector element such as a metal detector is capable of perceiving the location of the metal component.

In certain embodiments, the system of the present invention is configured to be used with a specific portion of a patient's body such as a nasal cavity or a mouth. In certain embodiments, the device may be configured to maintain a patient's mouth in an open position to permit insertion of a tube, may function as a bite block that protects the tube from the patient's teeth or mouth secretions, or may facilitate better oral care.

Embodiments of a device according to the present invention may permit proper tube placement using tubes with decreased rigidity compared with tubes not placed using a device according to the present invention. Since many embodiments of a device guide the tube toward its destination or control the entire path of the tube, tubes need relatively less rigidity to reach the desired position in the cavity of the patient than otherwise needed without a device.

Embodiments of the present invention decrease the discomfort experienced by a patient during the process of tube placement and while the tube and device are positioned in the patient's body. Advantageously, patients are likely to have an improved tolerance for leaving the tube in place for a longer period of time or for more frequent intubation when embodiments of the present invention are used. Embodiments of the present invention may decrease the needs for surgically placed feeding tubes, which are associated with increased costs and complications.

Embodiments of the present invention also decrease the number of placement attempts necessary to achieve desired placement of the device and tube. Advantageously, such embodiments permit the health care provider to deliver more timely treatment to the patient. The timeliness of treatment may be especially important, for example, when the tube will be used to remove a toxic substance that has been ingested or when the tube will be used to deliver oxygen, nutrition, or medication to the patient. In addition, by facilitating accurate and timely tube placement, embodiments of the present invention may lower the number of adverse effects such as damage to cavity, nosebleed, sinusitis, a sore throat, gagging, retching, sneezing, coughing, scarring, or tube misplacement. Moreover, by lowering adverse effects, the present invention may decrease the patient's recovery time and need for follow-up care by health care providers.

Advantageously, embodiments of the present invention also permit the health care provider to deliver more cost-effective treatment to the patient. By facilitating quick positioning of a tube, embodiments of the present invention permit a health care provider to spend less time positioning or replacing the tube and less time identifying the position of the tube through processes that may be time-consuming or expensive. Incorrect placement of tubes may require repeat placements, and repeat tests for verification of placement, leading to increased costs and exposure to hazards of testing, including radiation from X-ray tests.

In addition, certain embodiments of the present invention may add value to health care institutions in other ways. More specifically, certain embodiments permit proper placement of a tube or device with decreased pain and discomfort. Effective patient pain management often is associated with patients giving an institution a higher rating or recommendation. Such pain reducing embodiments also may result in fewer combative gestures from uncooperative patients, and accordingly, reduce potential for injury to health care providers.

In addition, embodiments of the present invention may allow nutritionally deficient patients to administer and receive feedings via an NG tube in the home, decreasing the need for and costs of hospitalizations and placement in skilled nursing facilities.

The present invention and its attributes and advantages will be further understood and appreciated with reference to the detailed description below of presently contemplated embodiments, taken in conjunction with the accompanying drawings.

### BRIEF DESCRIPTION OF THE DRAWINGS

FIG. 1 is a cross-sectional view of a pharynx;

FIG. 2A is a perspective view of an embodiment of a slider device in a collapsed state according to the present invention;

FIG. 2B is a perspective view of an embodiment of a slider device in an erected state according to the present invention;

FIG. 2C is a perspective view of an embodiment of a slider device in a collapsed state according to the present invention;

FIG. 2D is a perspective view of an embodiment of a slider device in a collapsed state according to the present invention;

FIG. 3 is a perspective view of an embodiment of a stylet device according to the present invention;

FIG. 4 is a perspective view of an embodiment of a director device according to the present invention;

FIG. 5A is a perspective view of an embodiment of a swallower device with deflated bladders according to the present invention;

FIG. 5B is a perspective view of an embodiment of a swallower device with inflated bladders according to the present invention;

FIG. 6A is a cross-section view of an embodiment of a slider device in a collapsed state according to the present invention;

FIG. 6B is a cross-section view of an embodiment of a slider device and a NG tube according to the present invention;

FIG. 6C is a cross-section view of an embodiment of a slider device in an erected state and a NG tube according to the present invention;

FIG. 7A is a cross-section view of an embodiment of a slider device with connector elements according to the present invention;

FIG. 7B is a cross-section view of an embodiment of a slider device and a NG tube according to the present invention;

FIG. 7C is a cross-section view of an embodiment of a slider device and a NG tube according to the present invention;

FIG. 7D is a cross-section view of an embodiment of a slider device in an erected state and a NG tube according to the present invention;

FIG. 8A is a cross-section view of an embodiment of a slider device with connector elements according to the present invention;

FIG. 8B is a cross-section view of an embodiment of a slider device and a NG tube according to the present invention;

FIG. 8C is a cross-section view of an embodiment of a slider device and a NG tube according to the present invention;

FIG. 8D is a cross-section view of an embodiment of a slider device in an erected state and a NG tube according to the present invention;

FIG. 9A is a perspective view of an embodiment of a director device according to the present invention;

FIG. 9B is a view from the proximal end of one embodiment of a director device according to the present invention;

FIG. 9C is a view from the distal end of one embodiment of a director device according to the present invention;

FIG. 10A is a perspective view of an embodiment of a device and tube according to the present invention;

FIG. 10B is a perspective view of an embodiment of a device and tube according to the present invention;

FIG. 10C is a perspective view of an embodiment of a device and tube according to the present invention;

FIG. 11A is a perspective view of an embodiment of a device according to the present invention;

FIG. 11B is a perspective view of an embodiment of a device and tube according to the present invention;

FIG. 11C is a perspective view of an embodiment of a device and tube according to the present invention;

FIG. 11D is a perspective view of an embodiment of a device according to the present invention;

FIG. 12A is a perspective view of an embodiment of a device and anchoring unit according to the present invention;

FIG. 12B is a perspective view of an embodiment of a device and anchoring unit according to the present invention;

FIG. 13A is a perspective view of an embodiment of a device and tube in which the device includes an arc element and a bite block;

FIG. 13B is a perspective view of another embodiment of a device and tube in which the device includes an arc element and a bite block;

FIG. 14A is a flowchart describing an embodiment of a method according to the present invention; and

FIG. 14B is a flowchart describing an embodiment of a method according to the present invention.

### DETAILED DESCRIPTION OF EMBODIMENTS OF THE INVENTION

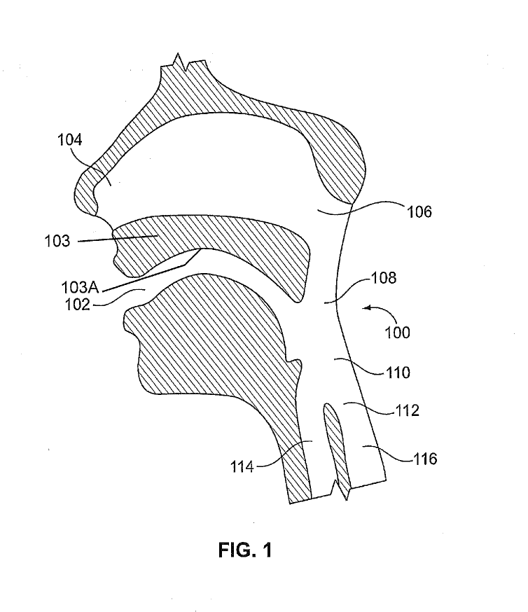

As shown in FIG. 1, the pharynx 100 is situated immediately posterior to the oral cavity or mouth 102 and nasal cavity or nostril 104. The pharynx 100 includes a nasopharynx 106, oropharynx 108, and hypopharynx 110. The nasopharynx 106 lies behind the nostril 104 and typically extends from the palate 103, including hard palate and soft palate, to the base of the skull. The lower edge of the palate 103 forms the roof of the mouth 103A. The oropharynx 108 lies behind the oral cavity 102. The hypopharynx 110, sometimes called the laryngopharynx, extends to the larynx 112, which is situated just below the junction that diverges into the trachea 114 and esophagus 116. The esophagus 116 leads to the stomach or, more generally, the abdomen. For purposes of this application, the term “pharynx” refers to the nasopharynx 106, oropharynx 108, hypopharynx 110, and sometimes larynx 112.

The system of the present invention includes various embodiments of an instrument for intubation including a NG tube 600 and a device 20 or a NG tube 600 paired with multiple devices 20. In embodiments in which the present invention includes an NG tube 600 and a device 20, the NG tube 600 may be positioned coaxially inside or outside of the device 20 or may be incorporated into the device 20. In certain embodiments, the NG tube 600 may be positioned side-by-side with the device 20 and, in such embodiments, the NG tube 600 and device 20 may be fixed to one another or may have no physical connection.

Embodiments of a NG tube 600 may include an insertion end 602 and a terminal end 604. A NG tube 600 also may include an inner tube surface 606 and an outer tube surface 608. The inner tube surface 606 may define a space 610 through which a substance may move.

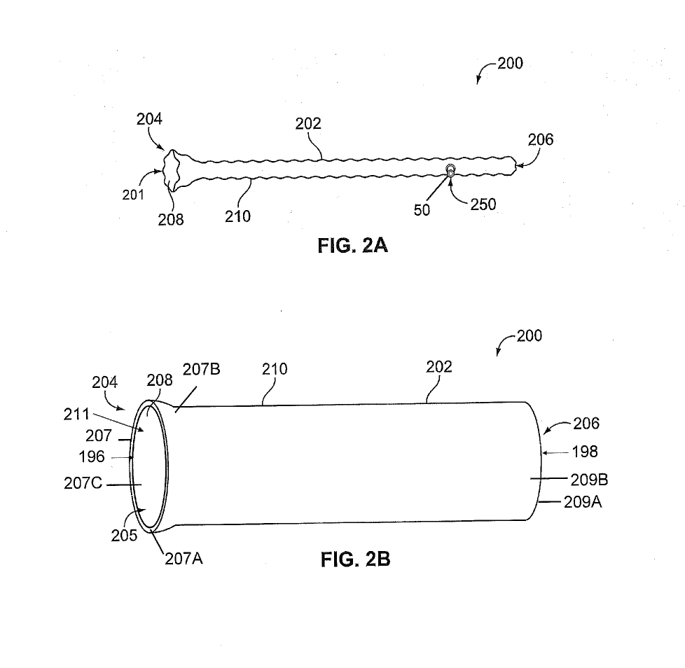

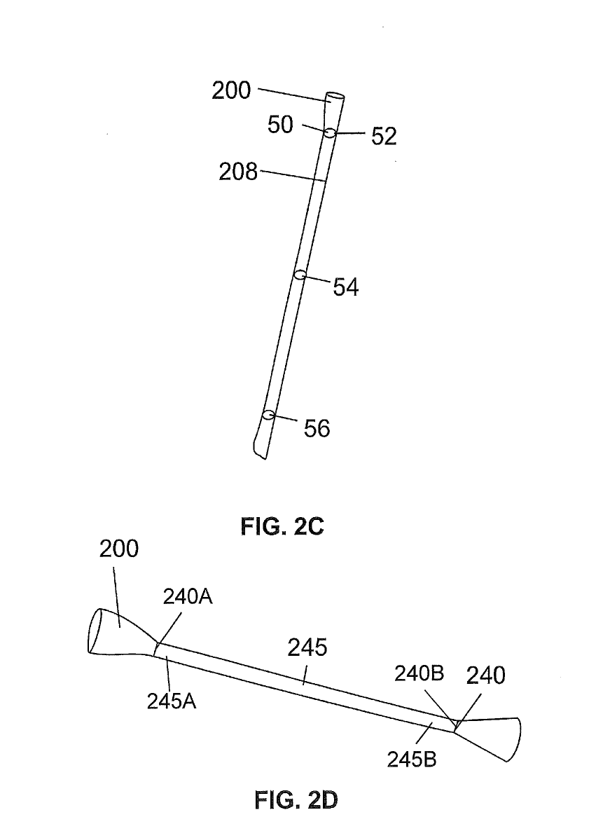

Embodiments of a device 20 may include a slider device 200 as shown in FIG. 2A and FIG. 2B. A slider device may include a body component 202. A body component 202 is also termed a “shaft component”, “tubular component”, or “conduit component” in this application. A body component 202 has a receiving end 204 and an input end 206. A receiving end 204 is also termed a “first end”, “proximal end”, a “near end”, or a “bottom end” in this application. An input end 206 is also termed a “second end”, “distal end”, a “far end”, or a “tip end” in this application. In certain embodiments, a receiving end 204 may include a receiving end surface 207 including a receiving end side surface 207A, a receiving end outer surface 207B, and a receiving end inner surface 207C. In certain embodiments, an input end 206 may include an input end side surface 209A, input end outer surface 209B, and input end inner surface (not shown). In certain embodiments, a receiving end side surface 207A forms an inlet 196 sized and shaped to permit entry of a NG tube 600. An input end side surface 209A may form an outlet 198 sized and shaped to permit exit of a NG tube 600. The body component 202 may be cylindrical in shape with an inside surface 208 and an outside surface 210, but any shape that can be inserted into a cavity such as a nostril 104 and pharynx 100 of a patient is contemplated. In certain embodiments, the inside surface 208 defines a channel 211, which may be configured to permit the passage of an NG tube 600.

The slider device 200 may be made from any flexible material including, for example, plastic or rubber. The flexible material may further include porous properties to allow nasal secretions to lubricate the slider device 200. It is also contemplated that the flexible material may include a water activated lubricant, an outer layer of lubricant, or an anesthetic agent. Lubrication of the slider device 200 assists in inserting the device 200 into a nostril 104 and into the pharynx 100 of a patient. Lubricant may include a flavored lubricant or scented lubricant.

In one embodiment, a NG tube 600 may be inserted into the slider device 200 before the NG tube 600 and slider device 200 are inserted together into a cavity such as a nostril 104 of the patient. A NG tube 600 may be positioned within the slider device 200 by sliding the NG tube 600 into the body component 202.

In other embodiments, the input end 206 of the slider device 200 is inserted into a nostril 104 of a patient and in the pharynx 100 prior to insertion of the NG tube 600. The receiving end 204 may either be completely inserted into the nostril 104 or may be exposed outside the nostril 104. In this embodiment, the slider device 200 is inserted into the patient in a collapsed state, as shown in FIG. 2A. Upon disruption of any constricting components 50, the slider device 200 may achieve an erected state as shown in FIG. 2B. Certain embodiments of a slider device 200 may include more than one constricting component 50—such as a first constricting component 52, a second constricting component 54, and a third constricting component 56—as shown in FIG. 2C.

In one embodiment, the slider device 200 is erected by threading the insertion end of a NG tube 600 through the receiving end 204 of the body component 202, and through the input end 206. In another embodiment, the receiving end 204 includes a lumen end 201 configured to maintain an open position to facilitate the insertion of a NG tube 600 or device 20 into the collapsed state of the slider device 200. The lumen end 201 is desirable in embodiments wherein the receiving end 204 of the slider device 200 is exposed, such as when a patient has a deviated septum within the nose or constricted areas within the pharynx 100.

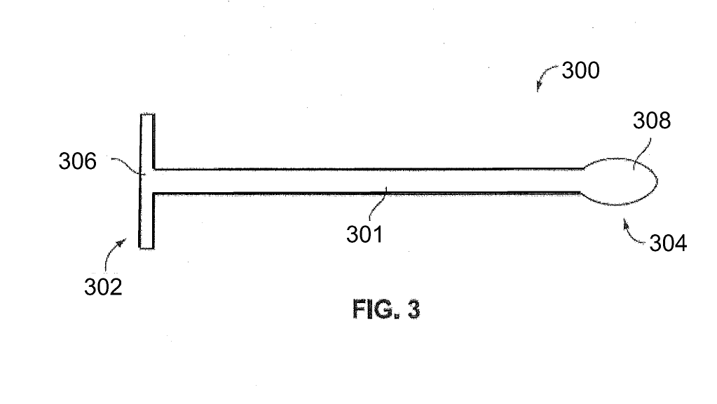

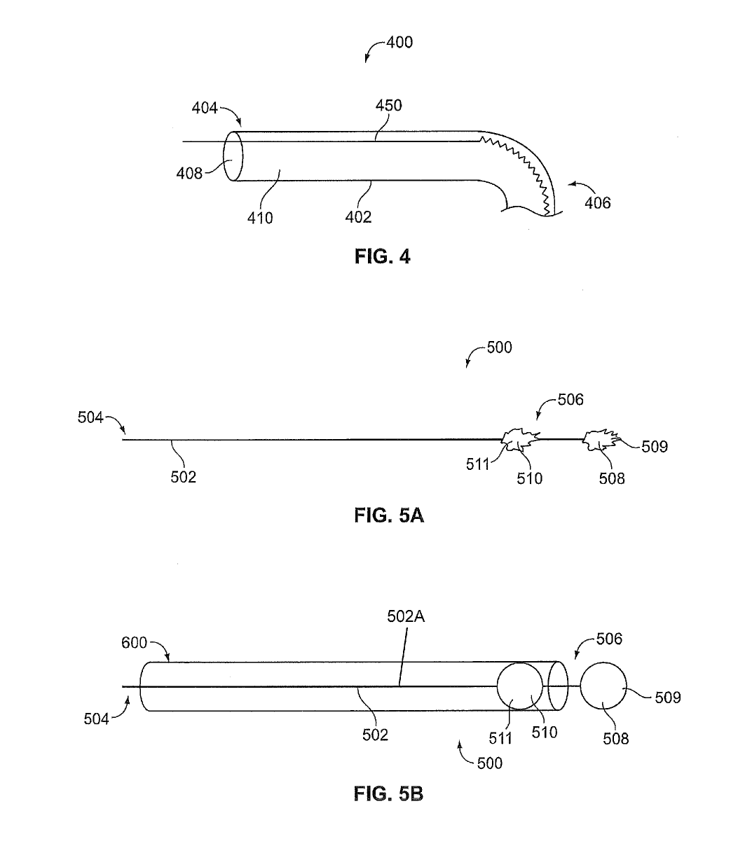

In one embodiment, the slider device 200 may be used in conjunction with the stylet device 300 discussed more fully in reference to FIG. 3. In another embodiment, the slider device 200 may be used in conjunction with the director device 400 discussed more fully in reference to FIG. 4. In yet another embodiment, the slider device 200 may be used in conjunction with the swallower device 500 discussed more fully in reference to FIG. 5. Generally devices 20 used together may be positioned side-by-side or coaxially relative to one another.

In certain embodiments, the slider device 200 may be maintained in a collapsed state using constricting components 50 such as seal elements 240, as shown in FIG. 2D. Certain embodiments of the seal elements 240 are attached to the inside surface 208 of a slider device 200 and define a suction compartment 245. A suction compartment 245 may be formed, for example, by compressing the slider device 200 and providing a seal at or near a first compartment end 245A and a second compartment end 245B to form a partial vacuum between a first seal element 240A and the second seal element 240B. In certain embodiments, the partial vacuum may be formed by suctioning air out of the suction compartment 245 before sealing with seal elements 240.

When a seal element 240 defining one end of the suction compartment 245 is broken, for example, by a NG tube 600 or device 20, the suction compartment 245 is breached and the slider device 200 may expand to achieve an erected state. In other embodiments, a seal element 240 may include a seal release element 244 such that the seal release element 244 may be used to break the seal element 245 and breach the suction compartment 245, thereby releasing the partial vacuum state and causing the slider device 200 to achieve an erected state. Embodiments of a seal release element 244 may include a pull-tab positioned relative to the seal element 240, string or rod bound to surface of seal element 240, or any other element configured to permit breaching the suction compartment 245 and releasing the partial vacuum state formed in the suction compartment 245.

In certain embodiments, a constricting component 50 includes a weak adhesion element 250 positioned on the inside surface 208 of a slider device 200 to maintain the collapsed state, such as a constricted state, shown in FIG. 2A. When a NG tube 600 is threaded through the slider device 200, the weak adhesion element 250 breaks such that the slider device 200 may achieve the erected state shown in FIG. 2B. In certain embodiments, the weak adhesion element 250 may be include any appropriate means of connection known to those skilled in the art, for example, adhesive or spot weld.

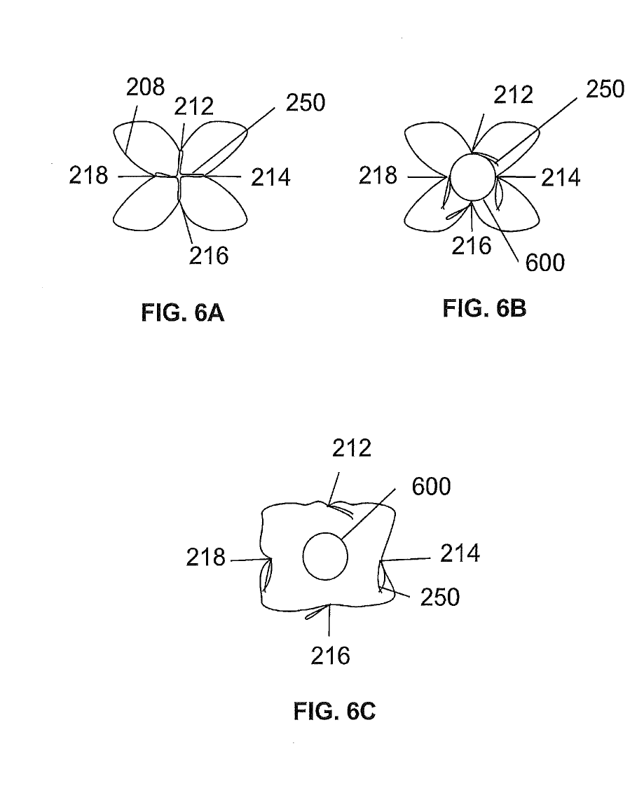

A weak adhesion element 250 may be positioned to adhere a portion of the inside surface 208 to another portion of the inside surface 208 of the body component 202. In the embodiment shown in FIG. 6A, a weak adhesion element 250 adheres to the inside surface 208 at a first inside point 212, a second inside point 214, a third inside point 216, and a fourth inside point 218. Certain embodiments may include a single weak adhesion element 250 that adheres to the inside surface 208 along the entire length of the slider device 200 or may include a weak adhesion element 250 that extends less than the entire length of the slider device 200. In certain embodiments, a number of discrete weak adhesion elements 250 may be positioned along part or all of the length of the slider device 200. In certain embodiments, a weak adhesion element 250 adheres to the inside surface 208 at a set of points that define part of or the entire inner circumference of the body component 202. Generally, the introduction of the NG tube 600 may break the weak adhesion element 250, as shown in FIG. 6B, and the body component 202 may achieve an erected state, as shown in FIG. 6C.

In certain embodiments, a constricting component 50, such as connector element 260, may connect a portion of the outside surface 210 to another portion of the outside surface 210 of the body component 202. Embodiments of a connector element 260 may connect the outside surface 210 at a first outside point 220, a second outside point 222, a third outside point 224, and a fourth outside point 226. A connector element 260 may include any appropriate means of connection known to those skilled in the art, for example, spot weld or a bonding agent. A connector element 260 may connect the entire length of the slider device 200 or may connect less than the entire length of the slider device 200. Certain embodiments of the present invention include multiple discrete connector elements 260.

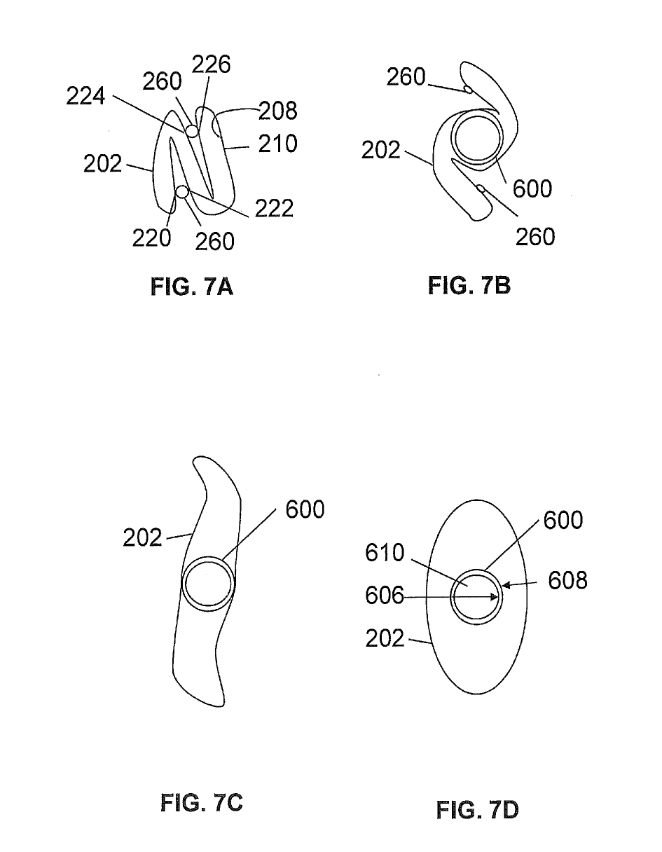

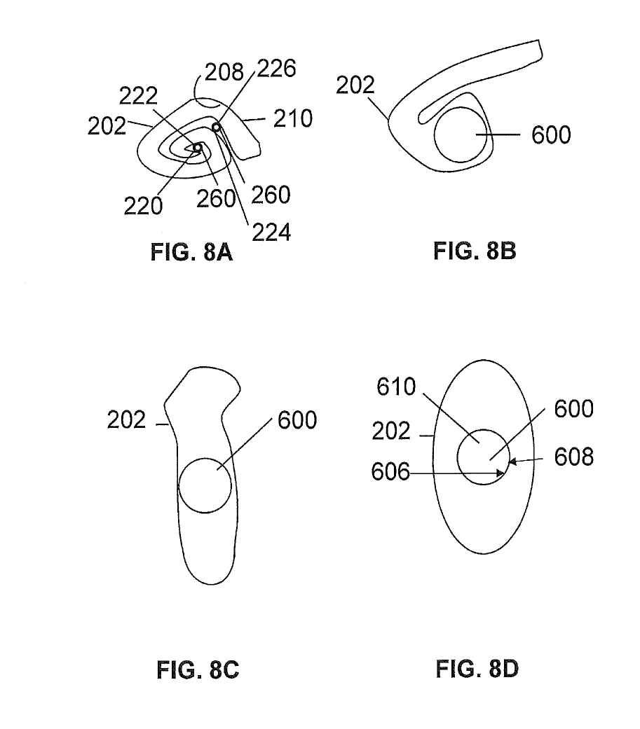

Embodiments of a connector element 260 may be configured to permit a slider device 200 to achieve a collapsed state, which may be maintained for a period of time. Certain embodiments of a collapsed state include a folded state, as shown in FIG. 7A through FIG. 7D, or a rolled-up state, as shown in FIG. 8A through FIG. 8D. In FIG. 7A and FIG. 8A, embodiments of a slider device 200 are illustrated in a collapsed state. In FIG. 7B and FIG. 8B, embodiments of a slider device 200 are illustrated in a first intermediate state, and in FIG. 7C and FIG. 8C, a slide device 200 are illustrated in a second intermediate state. In FIG. 7D and FIG. 8D, embodiments of a slider device 200 are illustrated in the erected state.

The stylet device 300, as shown in FIG. 3, includes a shaft component 301 having a near end 302 and a far end 304. The shaft component 301 is generally cylindrical in shape, but any small cross-sectional shape that can be inserted into the nostril 104 and in the pharynx 100 of a patient is contemplated. The shaft component 301 of the stylet device 300 may be made from any flexible material, such as plastic or rubber.

The near end 302 of the stylet device includes a handle element 306. In one embodiment, the far end 304 of the stylet device 300 includes a pellet element 308. The pellet element 308 may be made from any flexible material, such as plastic or rubber. The pellet element 308 may be spherical in shape, but any shape is contemplated that may be inserted into the cavity such as the nostril 104 and the pharynx 100 of a patient. Further, the pellet element 308 may be weighted to assist in positioning the stylet device 300.

The far end 304 of the stylet device 300 is inserted into the nostril 104 and in the pharynx 100 of a patient such that the near end 302 is exposed outside the nostril. A NG tube 600 or device 20 may be inserted into the nostril 104 and in the pharynx 100 of a patient using the stylet device 300 as a guide. For example, a NG tube 600 can be guided by the stylet device 300 when the NG tube 600 is inserted within the shaft component 301 prior to insertion into the nostril 104 and in the pharynx 100 of a patient. In another embodiment, the stylet device 300 may be inserted into the nostril 104 and in the pharynx 100 of a patient and then the NG tube 600 may be inserted into the nostril 104 and in the pharynx 100 to follow alongside the shaft component 301. In another embodiment, the stylet device 300 may be used in conjunction with the director device 400 discussed more fully in reference to FIG. 4. In another embodiment, the stylet device 300 may be used in conjunction with the swallower device 500 discussed more fully in reference to FIG. 5.

The director device 400, as shown in FIG. 4, includes a tubular component 402 having a proximal end 404 and a distal end 406. The tubular component 402 may be cylindrical in shape with an interior surface 408 and an exterior surface 410, but any shape that can be inserted into the nostril 104 and in the pharynx 100 of a patient is contemplated. The proximal end 404 may include various surfaces such as a proximal end surface 412, proximal end exterior surface 414, and proximal end interior surface 416. The distal end 406 may include various surfaces such as a distal end surface 418, distal end exterior surface 420, and distal end interior surface 422. The tubular component 402 of the director device 400 may be made from any flexible material, such as plastic or rubber.

The proximal end 404 of a director device 400 includes a guide element 450 made from any flexible material such as metal, plastic, or rubber that passes through the interior surface 408 and attaches at or near the distal end 406. The guide element 450 may attach to the interior surface 408 of the tubular component 402 by any locking means known to those skilled in the art, for example, adhesive.

In one embodiment, the distal end 406 of the director device 400 is inserted into the nostril 104 and in the pharynx 100 of a patient such that the proximal end 404 is exposed outside the nostril 104. The guide element 450 then may be manipulated, for example, to curve or angle the distal end 406 downward towards and past the pharynx 100, specifically the oropharynx 108.

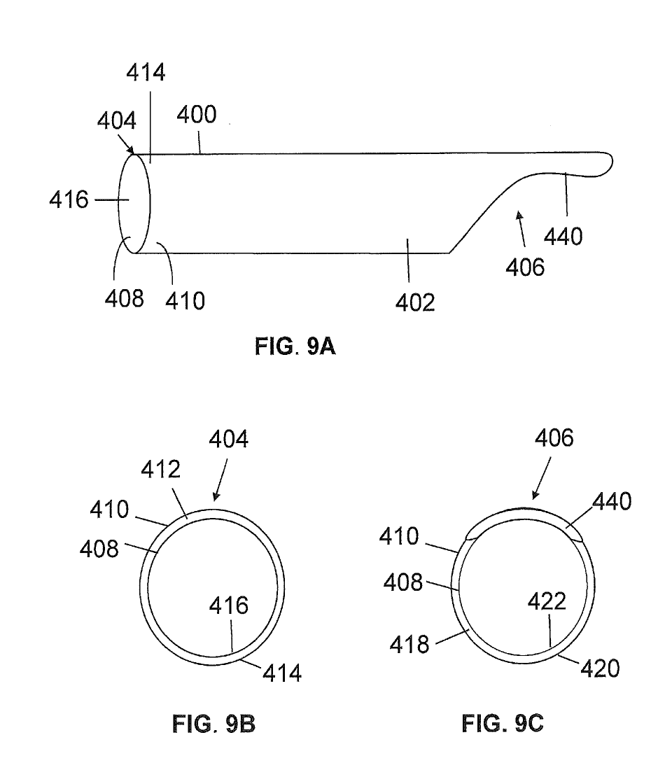

In another embodiment, the distal end 406 of the director device 400 includes a protrusion element 440 such as a tapered tip, as shown in FIGS. 9A-9C, that extends from the tubular component 402. A protrusion element 440 may provide leverage to open a patient's mouth when inserted into a mouth. A protrusion element 440 also may be manipulated, such as rotated or pulled, to curve or angle the distal end 406 of the director device 400 such that the NG tube 600 inserted within is directed along the curvature of a patient's pharynx 100, esophagus 116, or other internal structure.

Certain embodiments of a director device 400 additionally may include a control element. A control element permits a user to control the position of the guide element 450. Embodiments of a control element include a lever, switch, or knob at or near the distal end 406 of the director device 400 to facilitate the manipulation of the protrusion element 440.

Upon positioning the director device 400, a NG tube 600 or a device 20 is inserted through the interior surface 408. In one embodiment, the director device 400 may be used in conjunction with the swallower device 500 discussed more fully in reference to FIG. 5. In another embodiment, the director device 400 may be used in conjunction with the slider device 200 along with the stylet device 300.

The swallower device 500, as shown in FIGS. 5A and 5B, includes a conduit component 502 that includes a bottom end 504 to a tip end 506. The conduit component 502 is illustrated in a cylindrical shape, but any shape that can be inserted into the cavity such as a nostril 104 and the pharynx 100 of a patient is contemplated. The conduit component 502 of the swallower device 500 may be made from any flexible material, such as plastic or rubber.

In certain embodiments, a first bladder element 508 and a second bladder element 510 are positioned near the tip end 506 of the conduit component 502.

Each of the bladder elements 508, 510 are made from any flexible material, such as plastic or rubber. The first bladder element 508 may include a first opening 509. The second bladder element 510 may include a second opening 511. In certain embodiments, the bladder elements 508, 510 may be integrated into the conduit component 502 such that a substance positioned in the conduit component 502 may pass into the bladder elements 508, 510. In such embodiments, the bladder elements 508, 510 may inflate when injected with a selected substance, such as air, saline, or water as shown in FIG. 5B. The selected substance may be injected into the bladder elements 508, 510, for example, via a syringe inserted into the bottom end 504 of the conduit component 502. In other embodiments, the bladder elements 508, 510 may not be integrated into the conduit component 502, but may be linked to the outside surface 502A of the conduit component 502. In such embodiments, the bladder elements 508, 510 are configured to be inflatable by a syringe entering the bladder element 508, 510 directly or in any other manner known in the art.

Prior to injection, the bladder elements 508, 510 are in a deflated state as shown in FIG. 5A. A swallower device 500 may be inserted into the nostril 104 of a patient without other devices. In certain embodiments, a NG tube 600 or a device 20 is positioned over the conduit component 502 such that the first bladder 508 extends outside the NG tube 600 or device 20 and the second bladder 510 remains inside the NG tube 600 or device 20 as shown in FIG. 5B.

After positioning the NG tube 600 about the swallower device 500, the second bladder element 510 is inflated to secure the NG tube 600. The tip end 506 of the swallower device 500 is inserted into the nostril 104 and in the pharynx 100 of a patient. The depth to which the first bladder 508 should initially be inserted in the oropharynx 108 can be estimated by measuring the distance from the patients' nasolabial fold to the patient's tragus.

Upon the tip end 506 located beyond the nasopharynx 106 and into the oropharynx 108, the first opening 509 of the first bladder element 508 is inflated to a selected diameter such as 1 to 1.2 centimeters to provide the patient with the sensation that a food bolus is in his or her pharynx 100 to assist the patient in advancing the swallower device 500 including NG tube 600 into the stomach. If inflation causes sensation of high pressure within the nose, the bladder element may be deflated, advanced, and re-inflated. If the inflation causes pressure on the area posterior to the oropharynx 108, the bladder can be deflated, withdrawn approximately one centimeter and then inflated again.

When the bladder element 508 is inflated in the proper position, the patient is advised to swallow, possibly while drinking a liquid to aid in swallowing, which should begin to advance the NG tube 600 and swallower device 500 down the patient's esophagus 116. As the patient swallows, a health care provider may apply gentle pressure to the NG tube 600 and bladder element 508 to advance the NG tube 600.

Once the swallower device 500 and NG tube 600 are located within the stomach, the first bladder element 508 and the second bladder element 510 may be deflated. Deflation may occur by retracting the selected substance via the syringe that is inserted into the bottom end 504 of the conduit component 502 which permits removal of the swallower device 500 while allowing the NG tube 600 to remain. In other embodiments, deflation may occur by causing an opening such as a first opening 509 in the first bladder element 508 and a second opening 511 in the second bladder element 510. In such embodiments, the openings 509, 511 may be caused by a syringe which also extracts the selected substance from the first bladder element 508 and the second bladder element 510. In other embodiments, the first opening 509 and second opening 511 may permit the release of the selected substance into the patient's body. In such embodiments, the selected substance may be dissolvable or digestible such that it does not harm the patient.

In certain embodiments, the first bladder element 508 may either remain inflated or be re-inflated with air such that gastric peristalsis aids in maintaining the position of the NG tube 600 in the stomach. In another embodiment, the first bladder element 508 remains inflated or is re-inflated with a liquid to achieve a weight sufficient to urge the first bladder element 508 and accompanying NG tube 600 into the small intestine.

In another embodiment, a slider device 200 may be used in conjunction with a bladder element 508. In such an embodiment, a bladder element 508 is positioned within the inside surface 208 of a slider device 200 and may be inserted into the nostril 104 together. When the bladder element 508 and the slider device 200 reach a selected position, the bladder element 508 may be inflated by injecting a selected substance such as air, saline, or liquid to expand the bladder element 508. Expansion of the bladder element 508 may disrupt any constricting components 50 that are maintaining the slider device 200 in a collapsed state. The disruption of the constricting components 50 causes the slider device 200 to achieve an erected state.

In certain embodiments, the swallower device 500 may be used in conjunction with the director device 400. In another embodiment, the swallower device 500 may be used in conjunction with the slider device 200 along with the director device 400. In another embodiment, the swallower device 500 may be used in conjunction with the slider device 200 along with the stylet device 300. In yet another embodiment, the swallower device 500 may be used in conjunction with the slider device 200 along with the stylet device 300 along with the director device 400.

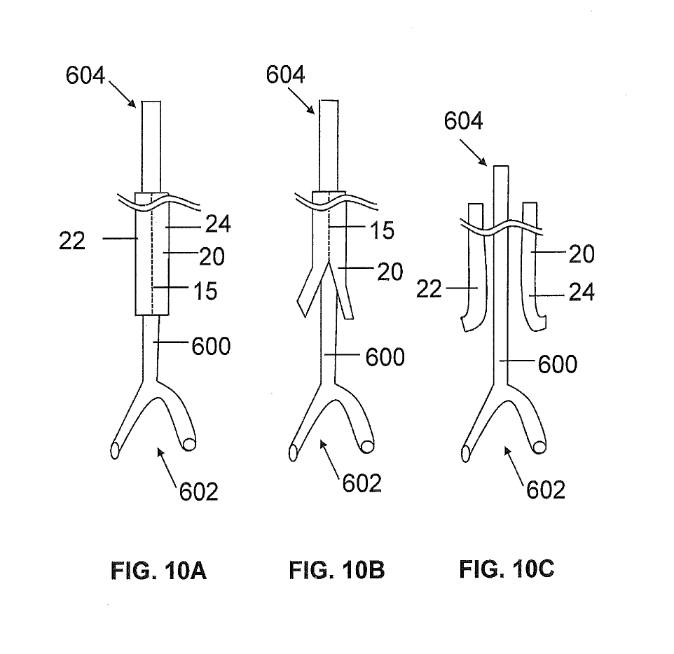

The devices 20 may be removed after positioning the NG tube 600. In certain embodiments, devices 20 may be removed by sliding the device 20 out over the NG tube 600. However, as shown in FIG. 10, an insertion end 602 may be configured as a wide end or an insertion end 602 having more than one outlet. Such embodiments of a device 20 do not permit easy removal of the device 20 by sliding over the NG tube 600. Certain embodiments of the devices 20 may be removed by separating the device 20 into a first side piece 22 and a second side piece 24 and then removing each piece. Devices 20 may be separated into pieces by, for example, breaking along a perforation 15 or tearing or cutting the material of the device 20.

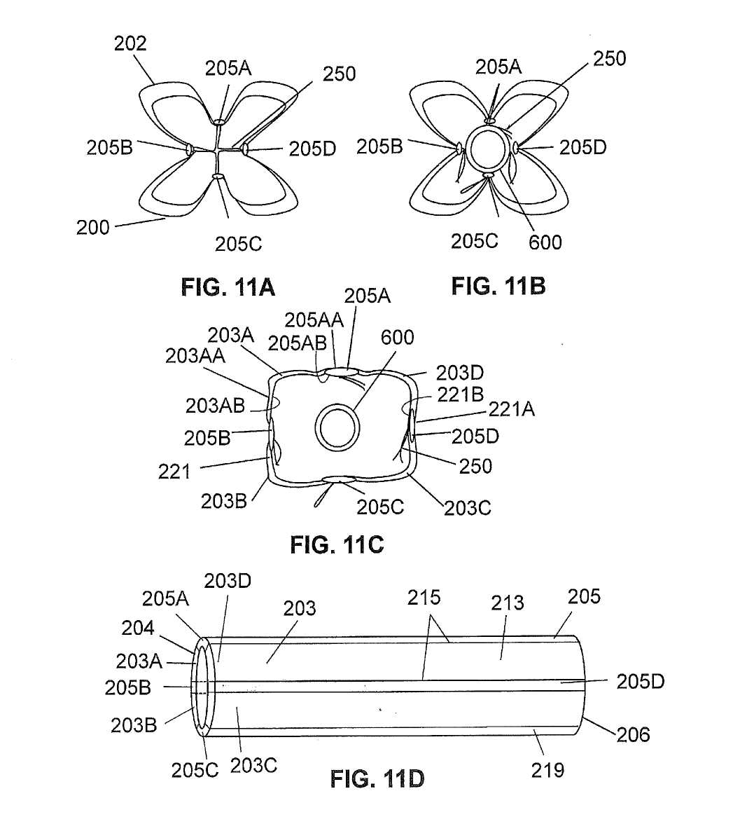

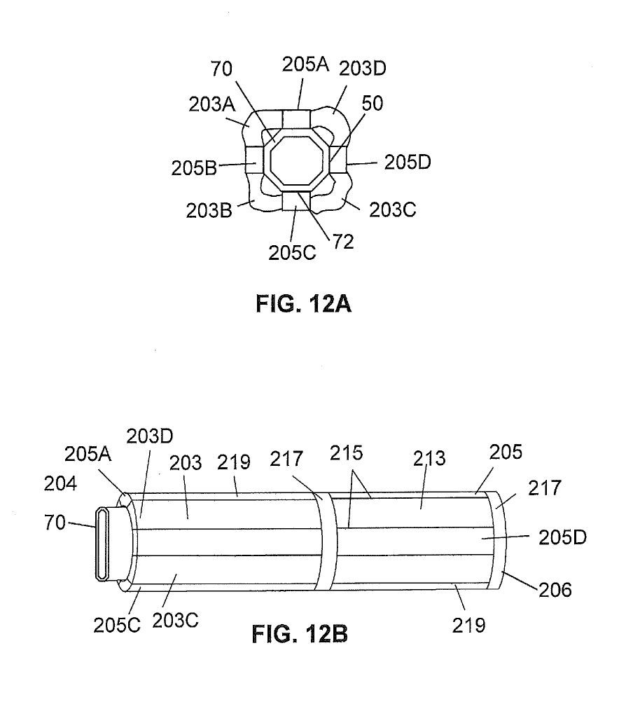

As shown in the embodiments shown in FIG. 11A-FIG. 11D, a slider device 200 may include a body component 202 comprised of two or more materials. In certain embodiments, one section of material may be a pliant body component 203 configured to be pliable and flexible in size, shape, and contour. Another section of material may include a bendable body component 205 which is relatively less flexible than the pliant body component 203 and is configured to confer sufficient structural strength to the body component 202 such that the body component 202 may be advanced through a patient cavity by putting pressure on the receiving end of the device 200. The bendable body component 205 also is configured to be flexible enough to adjust to a shape of a patient's cavity. The embodiment shown in FIG. 11D includes a first pliant body component 203A, a second pliant body component 203B, a third pliant body component 203C, and a fourth pliant body component 203D. A bendable body component 205 may be positioned between each pliant body component 203. The illustrated embodiment includes a first bendable body component 205A, a second bendable body component 205B, a third bendable body component 205C, and a fourth bendable body component 205D. Certain embodiments of a body component 202 include only pliant body components 203 or only bendable body components 205.

In certain embodiments, each pliant body component 203 is fastened to a bendable body component 205 by a fastening element 215. In certain embodiments, a pliant body component 203 may be fastened to a bendable body component 205 in a side-by-side configuration. Such a fastening element 215 may include adhesive, welding, bonding agent, or any appropriate means of fastening the pliant body component 203 to the bendable body component 205 known in the art. Certain embodiments of a pliant body component 203 may be configured to form a pouch in which a bendable body component 205 may be positioned. Certain embodiments of a bendable body component 205 may include a generally annular shaped elements 217, a generally linear shaped elements 219, or any other shape to provide sufficient structure or framing to the device 20. A bendable body component 205 may be positioned relative to a pliant body component 203 in any configuration such that the bendable body component 205 provides sufficient structure for the device to be moved into a patient's body.

As shown in FIG. 11C, embodiments of each pliant body component 203 may include an outer pliant body surface 203AA and an inner pliant body surface 203AB. Embodiments of each bendable body component 205 may include an outer bendable body surface 205AA and an inner bendable body surface 205AB. In certain embodiments, the pliant body component 203 and bendable body component 205 may form a continuous device surface 221. In such embodiments, any outer pliant body surfaces 205AA and outer bendable body surfaces 203AA may be configured to form a continuous device outer surface 221A. Any inner pliant body surfaces 203AB and inner bendable body surfaces 205AB may be configured to form a continuous device inner surface 221B.

In certain embodiments, a bendable body component 205 extends from a receiving end 204 to an input end 206 of the body component 202. In other embodiments, a bendable body component 205 does not extend from the receiving end 204 to the input end 206. In certain embodiments, the pliant body component 203 extends from a receiving end 204 to an input end 206 of the body component 202. In other embodiments the pliant body component 203 does not extend from the receiving end 204 to the input end 206 of the body component 202.

In certain embodiments, a constricting component 50 is positioned relative to one or more bendable body components 205 to maintain the collapsed state, such as a constricted state, shown in FIG. 11A. When a NG tube 600 is threaded through the slider device 200, the weak adhesion element 250 breaks and the slider device 200 may achieve an erected state as shown in FIG. 11C. In certain embodiments, the weak adhesion element 250 may be an adhesive or spot weld that adheres, for example, a first bendable body component 205A to a second bendable body component 205B, third bendable body component 205C, and a fourth bendable body component 205D. The weak adhesion element 250 may adhere any number of bendable body components 205 to one another or may adhere any number of pliant body components 203 to one another.

In certain embodiments of the present invention, a constricting component 50 includes an anchoring unit 70 configured to fit within a slider device 200. In such embodiments, one or more points of the inside surface 208 of a slider device 200 may be coupled to the anchoring unit 70 thereby permitting the slider device 200 to maintain a collapsed state. Such embodiments may include a coupling component 72 configured to couple the anchoring unit 70 to the slider device 200. A coupling component 72 may couple the anchoring unit 70 to the slider device 200 along the entire length of the anchoring unit 70, the entire length of the slider device 200, a portion of the anchoring unit 70, or a portion of the slider device 200. In certain embodiments, an anchoring unit 70 is longer in length than the slider device 200, and in other embodiments, the anchoring unit 70 is shorter in length than the slider device 200.

In certain embodiments, the anchoring unit 70 is configured to be detachable from the slider device 200 such that upon detachment, the slider device 200 may achieve an erected state. The anchoring unit 70 may be decoupled in a number of ways. An anchoring unit 70 may be retracted out of the slider device 200. An anchoring unit 70 may be pushed into the patient's body if, for example, the anchoring unit 70 is made from a digestible material. An anchoring unit 70 may be dissolved by exposing the anchoring unit to a dissolving agent which is configured to dissolve the anchoring unit 70 but not the slider device 200.

In certain embodiments, an anchoring unit 70 may be coupled to a slider device 200 having a body component 202. In such embodiments, a body component 202 may include only a pliant body component 203 since the anchoring unit 70 may provide the structural support necessary to insert the body component 202 into the patient's cavity. In other embodiments, an anchoring unit 70 may be used in conjunction with a slider device 200 having a bendable body component 205 or a combination of pliant body components 203 and bendable body components 205.

Certain embodiments of a body component 202 may be made from a material having sufficient flexibility to align the body component 202 with any portion of the patient's mouth 102, nasal cavity 104, or other cavity. Certain embodiments of a body component 202 may be made from a material having sufficient strength and rigidity to sustain a specific contour without inner or outer influence, which will be termed a “contour sustaining embodiment” 199 for purposes of this application. A material having such strong properties also may protect any tube 600 positioned within the channel 211 of the body component 202. Certain contour sustaining embodiments 199 are configured to achieve a collapsed state and an erected state.

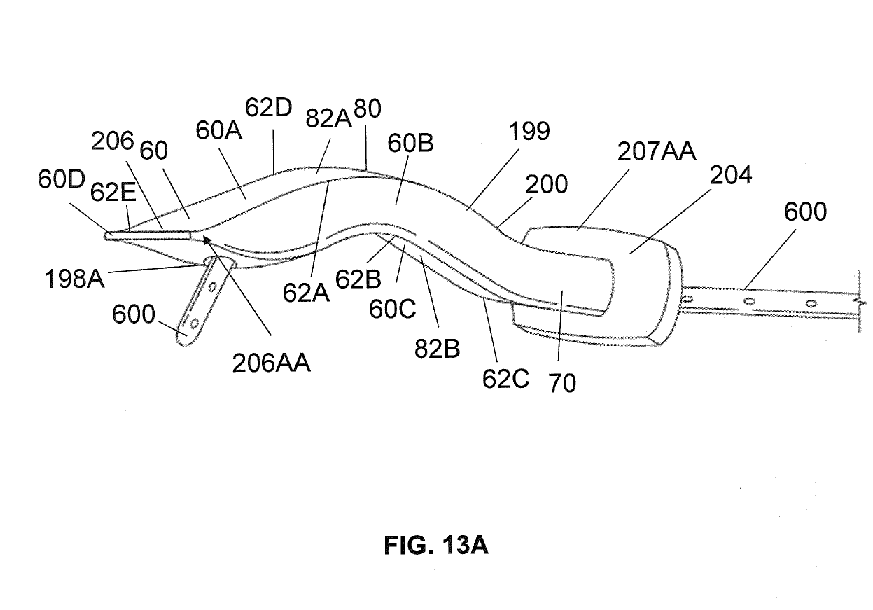

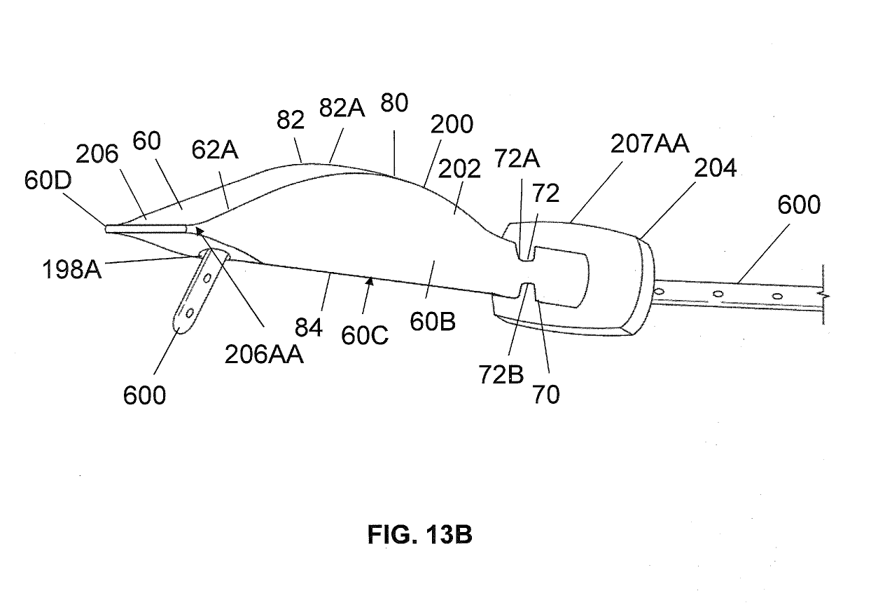

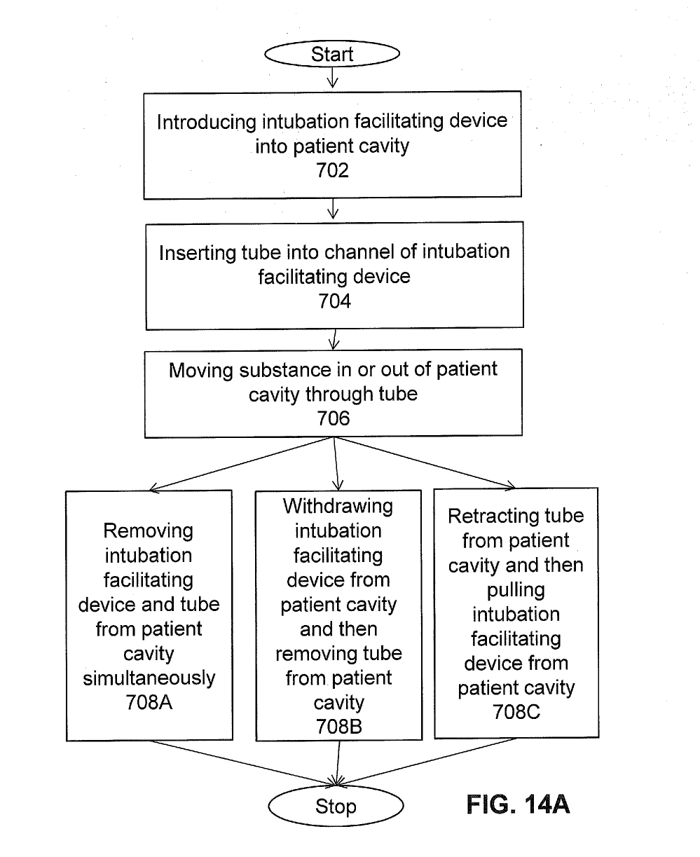

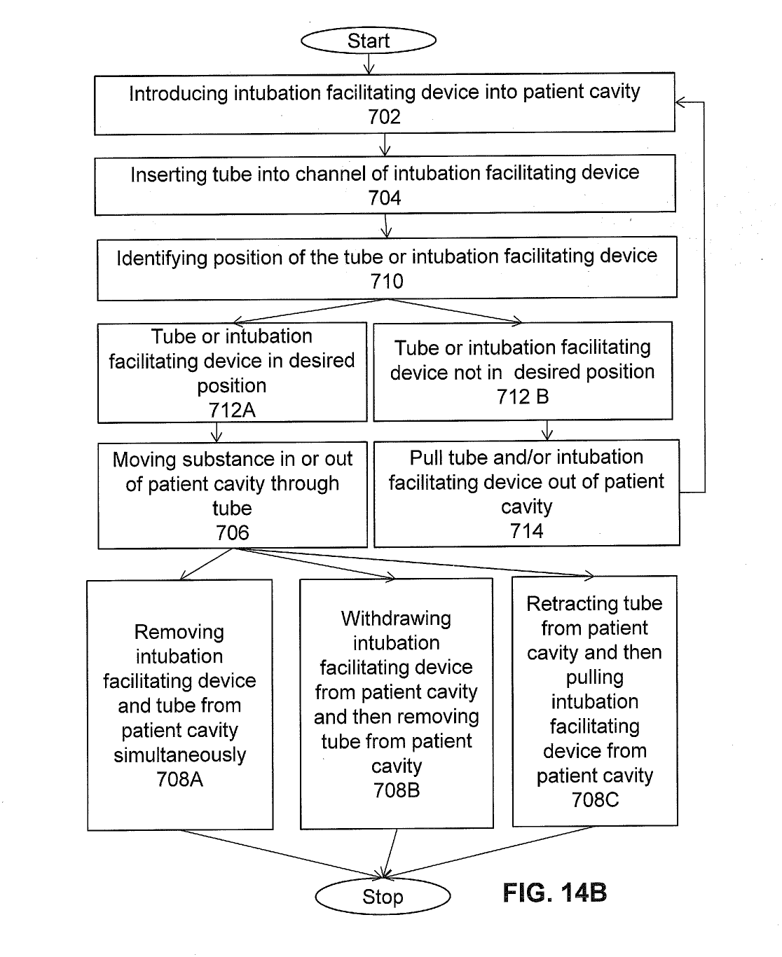

A contour sustaining embodiment 199 may include body component 202 having one or more walls 60 or edges 62. In certain embodiments, the body component 202 includes a continuous first wall 60A having no edges and having an inside surface 208 and an outside surface 210. In certain embodiments, a wall 60 include a first wall 60A, a second wall 60B, a third wall 60C, a fourth wall (not shown), and an insertion end wall 60D. In the embodiment illustrated in FIG. 13A, the first wall 60A meet a second wall 60B at a first edge 62A. The second wall 60B and a third wall 60C meet at a second edge 62B. The third wall 60C and a fourth wall (not shown) meet at a third edge 62C. The fourth wall (not shown) and a first wall 60A meet at a fourth edge 62D. The insertion end wall 60D meet the first wall 60A, second wall, 60B, third wall 60C, and fourth wall (not shown) at a rounded edge 62E. A contour sustaining embodiment 199 may be sized and shaped to be positionable adjacent to or inside of, for example, a portion of a patient's cavity. A contour sustaining embodiment 199 configured to be positionable into a patient's mouth 102 is illustrated in FIG. 13A and FIG. 13B. Such an embodiment may include a directing outlet 198A, a reduced-profile input end 206AA, a bite block 70, an arc element 80, or an expanded-profile receiving end surface 207AA.

In certain embodiments, an outlet 198 is positioned to influence the direction of movement of the tube 600 as the tube 600 exits the body component 202. One such embodiment is illustrated as a directing outlet 198A in FIG. 13A and FIG. 13B.

In certain embodiments, the wall 60 at or near the input end 206 has a reduced profile configured to be smaller in perimeter than other sections of a body component 202. Such an input end 206 will be termed “reduced profile input end area” 206AA for purposes of this application. A reduced profile input end 206AA may be sized and shaped to be positionable in a relatively narrow cavity or portion of a cavity, such as the posterior portion of the mouth 102.

Certain embodiments of a bite block 70 are configured to permit a patient to bite down on that section of the wall 60 without piercing the wall 60 or, in certain embodiments, without causing the wall 60 to change size or shape. A bite block 70 may be made from a material having sufficient strength or rigidity for the described purposes. Also, embodiments of a bite block 70 may include one or more dental acceptance elements 72 sized and shaped to receive at least a portion of the patient's teeth. Dental acceptance elements 72 also may be sized and shaped to receive the patient's gums or other section of the mouth. Certain embodiments include a first dental acceptance element 72A configured to receive the patient's upper teeth and a second dental acceptance element 72B configured to receive the patient's lower teeth. Other embodiments include only one dental acceptance element 72 or do not include a dental acceptance element 72.

Certain arc element 80 may be configured to provide leverage for opening an patient's mouth 102. In certain embodiments, an arc element 80 may be sized and shaped to be positionable adjacent to a portion of the patient's mouth 103A such as the roof of a patient's mouth 103A. In an arc element 80, one or more walls 60 form a curved surface 82; however, linear surfaces 84 also may be present in an arc element 80. In certain embodiments, such as the embodiment illustrated in FIG. 13A, an arc element 80 includes a curved top surface 82A and a curved bottom surface 82B. A curved top surface 82A is generally curved such that at least a portion of the curved surface 82 may be maintained in a raised position or lowered position relative to other portions of the wall 60, such as the topmost part of the receiving end 204 or the topmost part of the input end 206. A curved bottom surface 82B generally curved such that at least a portion of the curved surface 82 may be maintained in a raised position or a lowered position relative to other portions of the wall 60, such as the bottommost part of the receiving end 204 or the bottommost part of the input end 206.

A curved surface 82 also may be curved such that at least some portion of the curved surface 82 is maintainable in a raised position or lowered relative to a first side 65A or a second side 65B. A curved surface 82 also may include a generally flat plane 65AA extending from a first side 65A to a second side 65B, while sustaining a curve in another direction.

Each curved surface 82 in an embodiment may have the same curvature or have different curvature relative to one another. In the embodiment of FIG. 13A, the curved top surface 82A and the curved bottom surface 82B have similar arc shapes such that the first wall 60A and the third wall 60C are generally parallel and curved. Certain embodiments, such as the embodiment illustrated in FIG. 13B, include a curved top surface 82A and a generally linear bottom surface 84. Any combination of surfaces configured for insertion into a patient's cavity is contemplated.

Certain embodiments of a body component include an expanded-profile receiving end surface 207AA. An expanded-profile receiving end surface 207AA is configured to impede a body component 202 from advancing too far into a patient's cavity.

Embodiments of a method according to the present invention may include a number of steps. Such steps may include inserting a device into the cavity of a patient 702. Another step may include introducing the insertion end of the tube through the receiving end and into the channel of a device 704, a step that may occur before or after the device is positioned in the patient's cavity. The insertion end of the tube may be urged past the input end of the device if needed to properly position the tube. After the device and tube are in the desired position in the patient cavity, the health care provider may move a substance into or out of a patient cavity 706. In certain embodiments, the device may remain in the cavity until the substance transfer is complete. In such embodiments, the device and tube are removed simultaneously 708A. In other embodiments, the device is withdrawn while the tube remains in the patient cavity, and the tube is removed from the patient cavity after the device is withdrawn 708B. In such embodiments, the device may be separated into two or more pieces by tearing the slider device or breaking the slider device along a weakening such as a perforation and then withdrawing the pieces from the cavity. In other such embodiments, the device is withdrawn from the patient cavity without breaking it into pieces. In certain embodiments, the tube may be retracted from the patient cavity first and then the device may be pulled out of the patient cavity second 708C.

Another embodiment of a method according to the present invention may include the step of identifying the position of a tube or device 710 such as by performing an X-ray, conducting a pH test of the substance extracted from the patient cavity, or detecting an emission such as a sound, light, radio waves, magnetic field, or ultrasound waves caused by a detection component that corresponds with a tube or device. If the tube or device is in the desired position 712A, then a substance is moved in or out of the patient cavity through the tube 706. If the tube or device is not in the desired position 712B, the tube or device may be pulled out of the patient cavity 714 and the tube and device must be positioned again 702, 704.

Since certain embodiments of the present invention facilitate easier and more accurate positioning of the device and tube, such embodiments may decrease the cost of providing intubation services. More specifically, certain embodiments facilitate positioning a tube in a desired position in a single attempt or fewer attempts than conventional intubation apparatuses or processes. Accordingly, the health care provider likely spends less time positioning and identifying the position of the device and tube and likely conducts fewer expensive identifying steps such as taking an X-ray.

While the disclosure is susceptible to various modifications and alternative forms, specific exemplary embodiments have been shown by way of example in the drawings and have been described in detail. It should be understood, however, that there is no intent to limit the disclosure to the particular embodiments disclosed, but on the contrary, the intention is to cover all modifications, equivalents, and alternatives falling within the scope of the disclosure as defined by the appended claims.

What is claimed is:

1. An improved system for intubation of a patient, comprising:
a tube including an insertion end and a terminal end, said tube configured to facilitate transfer of a substance relative to a patient's body;
an intubation facilitating device including a receiving end, an input end, an outside surface, and an inside surface, said inside surface defining a channel which is constricted by a constricting component to achieve a collapsed state, said constricting component configured to maintain said intubation facilitating device in a collapsed state until said constricting component is disrupted by inserting said insertion end of said tube into said channel, whereupon said intubation facilitating device may achieve an erected state,
wherein, in said collapsed state, said intubation facilitating device achieves a low profile such that said intubation facilitating device may be inserted more easily and more comfortably into a cavity of the patient, said low profile maintainable before, during, and after said intubation facilitating device is positioned, and
wherein, in said erected state, said intubation facilitating device achieves an expanded profile such that said tube easily may be positioned through said channel.

2. The system of claim 1, wherein:
said inside surface of said intubation facilitating device includes a first inside point and a second inside point,
said constricting component includes a weak adhesion element by which said first inside point may adhere to said second inside point,
said weak adhesion element not positioned at or near said receiving end or said input end of said intubation facilitating device.

3. The system of claim 1, wherein:
said inside surface of said intubation facilitating device includes a first inside point and a second inside point,
said constricting component includes a weak adhesion element by which said first inside point may adhere to said second inside point, and
said weak adhesion element adheres to said inside surface of said intubation facilitating device along an entire length of said inside surface or almost the entire length of said inside surface, thereby constricting the entire length of said intubation facilitating device.

4. The system of claim 1, wherein:
said inside surface of said intubation facilitating device includes a first inside point, a second inside point, a third inside point, and a fourth inside point,
said constricting component includes a first weak adhesion element by which said first inside point is adhered to said second inside point and a second weak adhesion element by which said third inside point is adhered to said fourth inside point, wherein said first weak adhesion element and said second weak adhesion element are discrete and are sized, shaped, and positioned to constrict an entire length of said intubation facilitating device.

5. The system of claim 1, wherein said outside surface includes a first outside point and a second outside point and said constricting component includes a connector element by which said first outside point is connected to said first said second outside point.

6. The system of claim 1, wherein said constricting component is configured as one or more seal elements sized, shaped, and positioned to define a suction compartment in which a partial vacuum may be formed to maintain said intubation facilitating device in a collapsed state.

7. The system of claim 1, wherein said constricting component includes an anchoring unit and a coupling component, said coupling component configured to couple said inside surface of said intubation facilitating device to said anchoring unit such that said intubation facilitating device may achieve a collapsed state.

8. The system of claim 1, wherein said intubation facilitating device includes a pellet element at or near said input end of said device, said pellet element sized and shaped to be easily swallowed by a patient, thereby facilitating positioning of said intubation facilitating device in the patient.

9. The system of claim 1, wherein said intubation facilitating device includes a guide element configured to facilitate manipulating said input end of said intubation facilitating device, said guide element including a first guide end and a second guide end, and said first guide end is joined to said input end of said intubation facilitating device.

10. The system of claim 1, wherein said intubation facilitating device includes a detection component configured to cause an emission that is detectable from a location outside of the patient's body such that a position of said detection component inside of the patient's body may be identified.

11. The system of claim 1, wherein said intubation facilitating device includes a body component comprised of a bendable body component configured to confer sufficient structure to said body component such that said body component may be advanced into said cavity of the patient and a pliant body component configured to be more flexible than said bendable body component, wherein at least one of said bendable body component is fastened to at least one of said pliant body component such that said bendable body component and said pliant body component form a continuous device surface.

12. The system of claim 1, wherein said intubation facilitating device includes one or more perforations configured to facilitate separating said intubation facilitating device into two or more pieces, said perforations extending from said outside surface to said inside surface of said intubation facilitating device.

13. The system of claim 1, wherein either said intubation facilitating device or said tube includes a flavor coating.

14. The system of claim 1, wherein said intubation facilitating device is made from a material designed to permit said intubation facilitating device to transition from said collapsed state to said erected state upon a change in an environmental condition.

15. The system of claim 1 further comprising an erecting unit configured to disrupt said constricting component, whereupon said intubation facilitating device may achieve said erected state.

16. The system of claim 15, wherein said erecting unit includes a conduit component and one or more bladder elements which are positionable within said intubation facilitating device such that inflation of said one or more bladder elements causes disruption of said constricting component.

17. The system of claim 15, wherein said erecting unit includes a second intubation facilitating device including at least one from the following group of a intubation facilitating device configured to achieve a collapsed state and an erected state, an intubation facilitating device having a protrusion element, a intubation facilitating device having a pellet element, an intubation facilitating device having a guide element, and an intubation facilitating device having a bladder element.

18. An enhanced system for intubation of a patient, comprising:
a tube including an insertion end and a terminal end, said tube configured to facilitate transfer of a substance relative to a patient's body;
an intubation facilitating device including a receiving end, an input end, a wall having an outside surface and an inside surface, said inside surface defining a channel through which said tube may be positioned, said wall including at least a reduced profile input end area, an arc element, and a bite block,
said reduced profile input end area having a reduced profile relative to other portions of said intubation facilitating device,
said arc element including a curved surface, at least a portion of said curved surface maintainable in a raised positioned relative to said receiving end or said input end, said arc element configured to self-sustain said raised position, and
said bite block configured to permit a patient to bite down onto said bite block without teeth of the patient piercing said bite block.

19. An improved method for intubation of a patient, comprising the steps of:
inserting an input end of an intubation facilitating device into a cavity of the patient, said intubation facilitating device including said input end, a receiving end, an outside surface, and an inside surface, said inside surface defining a channel which is constricted by a constricting component,
wherein said constricting component is configured to maintain said intubation facilitating device in a collapsed state until said constricting component is disrupted by an insertion end of said tube;
introducing said insertion end of said tube through said receiving end and into said channel of said intubation facilitating device, thereby disrupting said constricting component and causing said intubation facilitating device to transition from the collapsed state to an erected state.

20. The method of claim 19, further comprising the step of:
producing a partial vacuum in a portion of said channel defined by said constricting component such that said intubation facilitating device is maintained in a collapsed state until said tube is inserted into said intubation facilitating device, said constricting component configured as a first seal element and a second seal element, wherein said portion of said channel between said first seal element and said second seal element forms a suction compartment.
